(19) United States
(12) Patent Application Publication
(10) Pub. No.: US 2021/0154202 A1
(43) Pub. Date: May 27, 2021

(54) SYNTHESIS OF DENTIMOL, AN ANTIGLAUCOMA DRUG

(71) Applicant: Virginia Commonwealth University, Richmond, VA (US)

(72) Inventors: Michael LANCINA, Richmond, VA (US); Juan WANG, Richmond, VA (US); Hu YANG, Glen Allen, VA (US)

(73) Assignee: Virginia Commonwealth University, Richmond, VA (US)

(21) Appl. No.: 17/048,586

(22) PCT Filed: Apr. 16, 2019
(86) PCT No.: PCT/IB2019/053155

§ 371 Date: Oct. 17, 2020

Related U.S. Application Data
(60) Provisional application No. 62/659,019, filed on Apr. 17, 2018.

(51) Int. Cl. A61K 31/5377; A61K 47/60; A61K 47/59; A61K 47/61; A61K 9/00; A61P 27/06

(52) U.S. Cl. CPC A61K 31/5377; A61K 47/60; A61P 27/06; A61K 47/61; A61K 9/0048; A61K 47/593

(57) ABSTRACT

Compounds, comprising a selective β-blocker coupled to a PAMAM dendrimer via a flexible spacer, and compositions containing the compounds, are provided. Methods of making and using the compounds and compositions for the treatment of glaucoma and other maladies are also provided.

### RELATED APPLICATIONS

This application claims the benefit of U.S. Application 62/659,019, filed Apr. 17, 2018, the entire contents of which are hereby incorporated by reference.

### GOVERNMENT SUPPORT

This invention was made with government support under Grant No. R01EY024072, awarded by the National Institutes of Health (NIH), and Support Grant No. P30CA016059 awarded by the NIH-NCI Cancer Center. The United States Government has certain rights in the invention.

### FIELD OF THE INVENTION

The invention provides compounds, which include a selective β-blocker coupled to a polyamidoamine (PAMAM) dendrimer via a flexible spacer, and compositions for the treatment of glaucoma and related diseases. In some embodiments, the selective β-blocker is timolol, the PAMAM dendrimer is polyamidoamine dendrimer generation 3.0, and the flexible spacer is polyethylene glycol.

### BACKGROUND

Ocular hypertension, i.e., elevated intraocular pressure (IOP), is characteristic of glaucoma, a chronic ocular disease that can lead to degenerative and irreparable vision loss. Although the pathophysiology leading to glaucoma is multifactorial, the immediate aim of treatment is to lower IOP back to normal levels and prevent disease progression. Pharmacologic therapies are usually the first treatment option because they are less costly and less invasive than surgical interventions. Beta-adrenergic antagonists, commonly known as β-blockers, reduce IOP by slowing the production of aqueous humor. Timolol maleate is a β-blocker developed in the 1970s. It selectively binds to adrenergic receptors in the ciliary body, making it a more potent IOP lowering drug than other β-blockers. Despite the existence of these potent IOP lowering drugs, glaucoma remains the second leading cause of blindness worldwide. This is because the impact of current treatments is severely limited by inefficient drug delivery formulations. The bioavailability of timolol delivered by traditional eye drops is very poor, requiring frequent dosing and suffering from poor patient compliance. For example, when timolol is delivered as a topical solution, only about 5% of the total drug at best makes it to the target organ with each dose. Because of this, the period after a dose where the drug is within its therapeutic concentration window in the anterior chamber is narrow, typically 4-5 hours. The remaining volume is flushed into systemic circulation where it can cause cardiovascular side effects of varying severity.

New antiglaucoma drugs are being actively pursued by targeting different therapeutic targets to acquire IOP reduction and neuroprotective benefits, for example via increasing optic nerve head blood flow or trabecular meshwork outflow.

Cationic dendrimers are known to readily penetrate mucus membranes and have been investigated as trans-corneal drug delivery vehicles in the past.

Polymeric vehicles that can improve the delivery efficiency of this drug could reduce either the necessary concentration or dosing regimen, representing a significant improvement in the clinical management of glaucoma. The study of nanoparticle vehicles to improve the bioavailability of topical ocular drugs has received considerable attention in the past decade. Most drug loss from eye drops occurs due to pre-corneal mechanisms. Generally, most drugs penetrate the corneal epithelium slowly relative to the rate of tear drainage and turnover. Polyamidoamine (PAMAM) dendrimers have the potential to overcome these factors because they have appropriate size and mucoadhesiveness to both slow washout and increase cornea permeability. PAMAM dendrimers have been complexed with other ocular therapeutics, resulting in improved drug delivery efficiency, but these methods rely on the formation of metastable dendrimer-drug complexes. In the past, however, no success has been reported in developing polymeric drugs for glaucoma.

### BRIEF DESCRIPTION OF THE FIGURES

FIG. 1. Chemical structures of common β-blockers (1), timolol (2), OTM (3), and one exemplary embodiment of the dendrimer-β-blocker conjugate compound, sometimes referred to herein as DenTimol.

FIG. 2. Synthesis of DenTimol.

FIG. 3. In vitro assessment of DenTimol. (A) Dose-dependent cytotoxicity of OTM and DenTimol. (B) Ex vivo permeation of OTM, OTM-PEG and DenTimol across rabbit cornea.

FIG. 4. In vivo IOP-lowering assessment of DenTimol in normotensive rats after one-time topical administration. * indicates p<0.05 vs. timolol PBS eye drops.

FIG. 5. Presents schematic example of development of a “smart” dendrimer carrier G4-acetal-NH₂ containing acid-labile acetal bonds that can change the positive surface charge to neutral at various rates in a weakly acidic to a neutral environment (pH5-7.4). G4-acetal-NH₂ and pH of aqueous humor changes from neutral to weakly acidic as a result of medication and pathogenesis of glaucoma. This new type still offers amine groups for dendrimer-β-blocker conjugate synthesis.

FIG. 6. Presents Zeta potential over time for an exemplary embodiment.

FIG. 7. Presents one exemplary synthesis of DenTimol. Either dendrimer G3 or G4-acetal-NH₂ can be used as underlying carrier.

FIG. 8 Presents a table of some exemplary β-blockers and their oxirane analogs.

### BRIEF DESCRIPTION OF THE SEVERAL EMBODIMENTS

One embodiment provides a compound, comprising a selective β-blocker coupled to a PAMAM dendrimer via a flexible spacer.

One embodiment provides a pharmaceutical composition, comprising the compound and a physiologically acceptable carrier.

One embodiment provides a method of treating ocular hypertension or glaucoma, comprising administering the compound alone or in combination with a physiologically acceptable carrier to a subject.

One embodiment provides a method, comprising carrying out the following reaction:

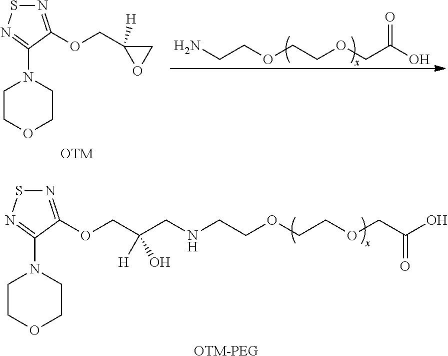

wherein x is 1-500.

One embodiment provides a method, comprising carrying out the following reaction:

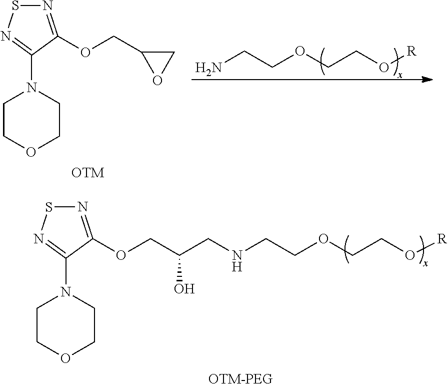

wherein x is 1-500; and
wherein R is one of the following:

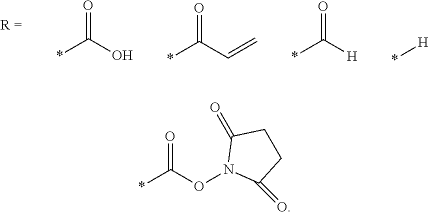

One embodiment provides a method, comprising carrying out the following reaction:

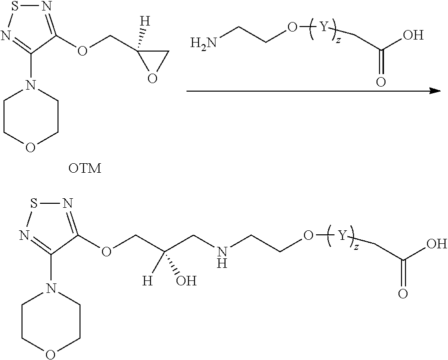

wherein Y is repeat unit of a flexible polymeric spacer; and
wherein z is 1-500.

One embodiment provides a method, comprising carrying out the following reaction:

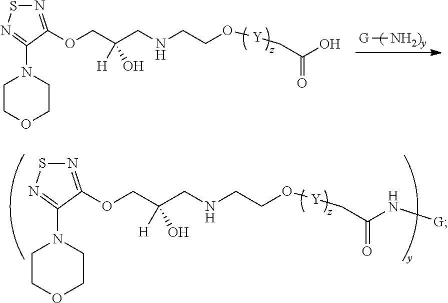

wherein Y is repeat unit of a flexible polymeric spacer;
wherein G is polyamidoamine dendrimer generation 2.0, 3.0, 4.0, or 5.0;
wherein y is 1-128; and
wherein z is 1-500.

One embodiment provides a method, comprising carrying out the following reaction:

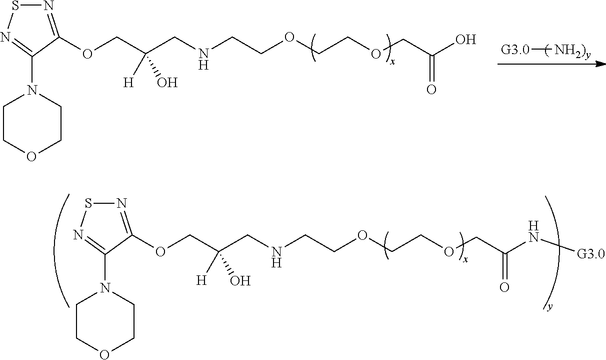

wherein G3.0 is polyamidoamine dendrimer generation 3.0;
wherein x is 1-500;
and wherein y is 1-32.

One embodiment provides a method, comprising carrying out the following reaction:

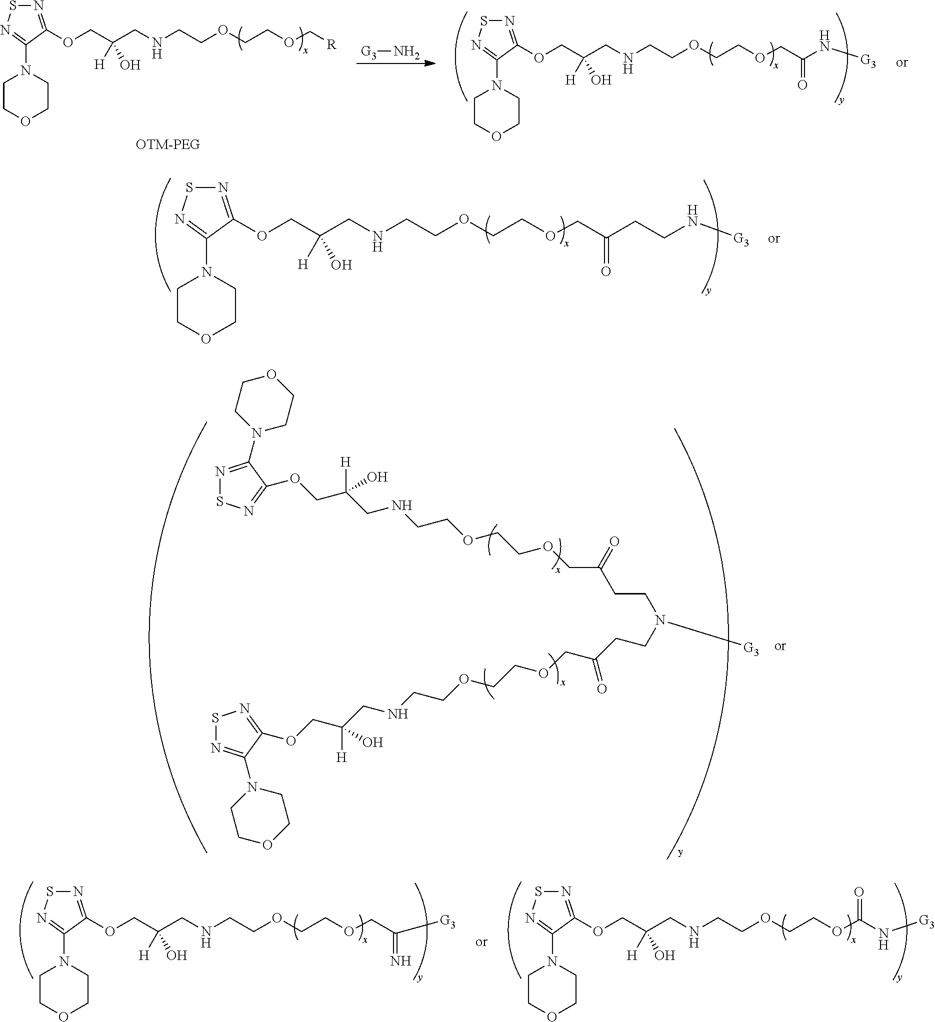

wherein G3.0 is polyamidoamine dendrimer generation 3.0;
wherein x is 1-500;
wherein y is 1-32; and
wherein R is one of the following:

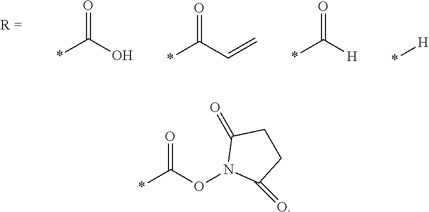

One embodiment provides a compound, having the following formula:

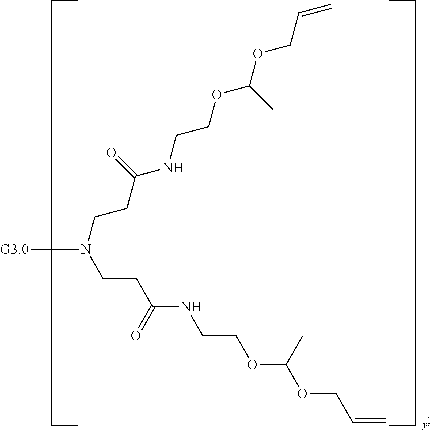

wherein G3.0 is polyamidoamine dendrimer generation 3.0;
and y is 1-32.

One embodiment provides a compound, having the following formula:

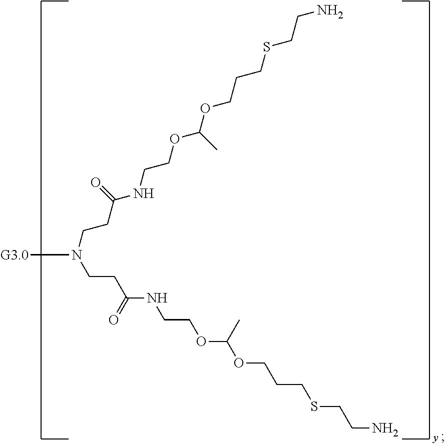

wherein G3.0 is polyamidoamine dendrimer generation 3.0;
and wherein y is 1-32.

One embodiment provides a compound, having the following formula:

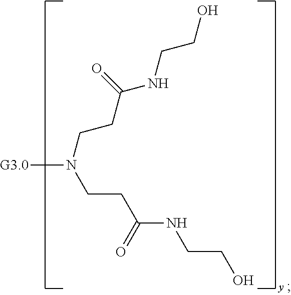

wherein G3.0 is polyamidoamine dendrimer generation 3.0;
and wherein y is 1-32.

One embodiment provides a method, comprising carrying out the following reaction:

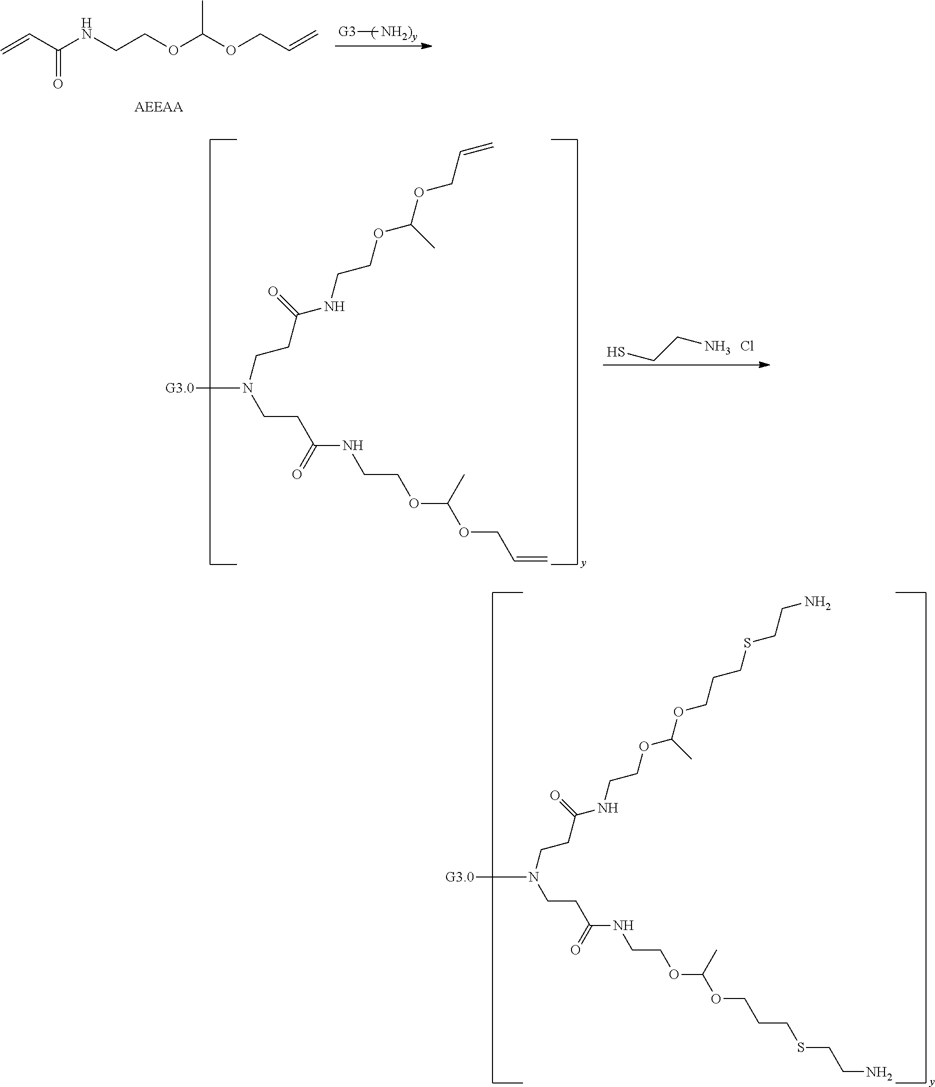

wherein G3.0 is polyamidoamine dendrimer generation 3.0;

and wherein y is 1-32.

One embodiment provides a method, comprising reacting a pharmaceutically active compound, comprising an oxirane or epoxide group, with a flexible spacer and a PAMAM dendrimer, to form a pharmaceutically active dendrimer.

### DETAILED DESCRIPTION OF THE SEVERAL EMBODIMENTS

In embodiments, the selective β-blocker is covalently bonded to the flexible spacer, and the spacer is covalently bonded to the PAMAM dendrimer.

In embodiments, the selective β-blocker is covalently coupled to the flexible spacer via a reaction of an oxirane group on the selective β-blocker.

In embodiments, the selective β-blocker is selected from the group consisting of Acebutolol, Atenolol, Betaxolol, Bisoprolol, Carteolol, Esmolol, Metoprolol, Nadolol, Penbutolol, Pindolol, Propranolol, Timolol, or combination thereof.

In embodiments, the selective β-blocker is timolol.

In embodiments, the PAMAM dendrimer is polyamidoamine dendrimer generation 2.0, 3.0, 4.0, or 5.0 (G2, G3, G4, or G5).

In embodiments, the flexible spacer is polyethylene glycol, polylactic acid, poly-L-lactic acid, poly(lactic-co-glycolic) acid, hyaluronic acid, or combination thereof.

In embodiments, the flexible spacer has one of the following formulas:

PLA (Polylactic acid), PLLA (Poly-L-lactic acid)

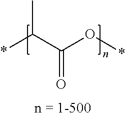

PLGA [Poly(lactic-co-glycolic acid)]

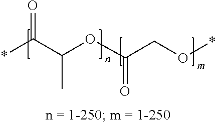

HA (Hyaluronic acid)

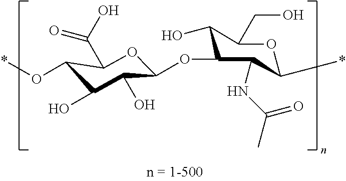

The ranges given for n and m independently include all values and sub-ranges therebetween. For example, for PLA and PLGA, n=1-500 includes 1, 10, 50, 100, 150, 200, 300, 400, and 500; for PLGA, n=1-250 includes 1, 10, 25, 50, 100, 150, 200, and 250, and m=1-250 includes 1, 10, 25, 50, 100, 150, 200, and 250; and, for HA, n=1-500 includes 1, 10, 50, 100, 150, 200, 300, 400, and 500.

In embodiments, the flexible spacer is hydrolyzable.

In embodiments, a plurality of the selective β-blockers may be covalently coupled to the PAMAM dendrimer via a plurality of respective flexible spacers.

In embodiments, the compound may have one of the following formulas:

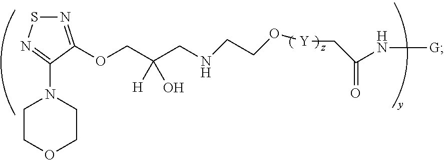

wherein Y is repeat unit of a flexible polymeric spacer;
wherein G is polyamidoamine dendrimer generation 2.0, 3.0, 4.0, or 5.0;
wherein y is 1-128; and
wherein z is 1-500.

In embodiments, the compound may have the following formulas:

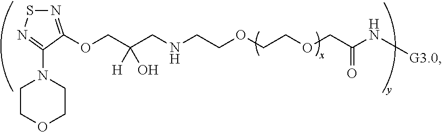

wherein G3.0 is polyamidoamine dendrimer generation 3.0;
wherein x is 1-500;
and wherein y is 1-32.

In embodiments, the compound may have one of the following formulas:

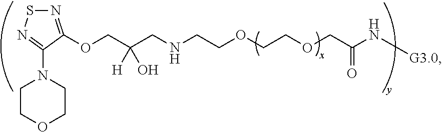

wherein G3.0 is polyamidoamine dendrimer generation 3.0;
wherein x is 44;
and wherein y is 22.

In embodiments, the physiologically acceptable carrier is an ophthalmic or ophthalmologically acceptable liquid carrier, solvent, diluent, emulsion, or dispersion. Non-limiting examples include saline, phosphate-buffered saline, water, polyethylene glycol, artificial tears, and the like.

In embodiments, the physiologically acceptable carrier is an ophthalmic or ophthalmologically acceptable solid carrier, for example, semi-solid and solid ophthalmic forms such as gels, ointments, contact lenses, ocular inserts, artificial tear inserts. Those carriers are made of a broad range of polymeric materials such as polyethylene glycol, polyvinyl alcohol, poloxamers, hyaluronic acid, carbomers, and polysaccharides.

The compound or composition may also be used in combination therapy. Combigan™ is a clinically prescribed combination product of brimonidine/timolol ophthalmic solution. The compound or composition can be administered together along with brimonidine and/or Combigan™ for a combination therapy.

In embodiments, the compound may have one of the following formulas:

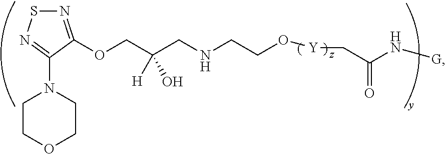

wherein Y is a flexible spacer;
wherein G is polyamidoamine dendrimer generation 2.0, 3.0, 4.0, or 5.0;
wherein y is 1-128; and
wherein z is 1-500.

In embodiments, x is 44 and y is 22 in the compound.

In embodiments, the compound may have the following formula:

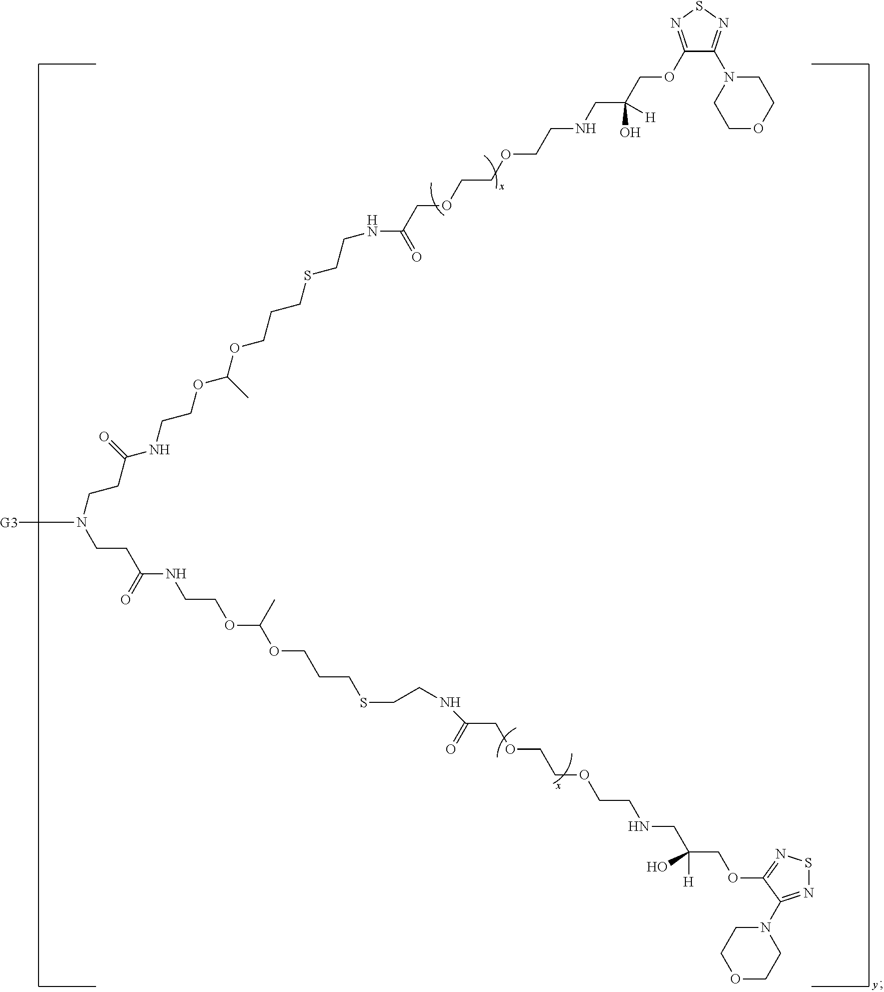

wherein G3.0 is polyamidoamine dendrimer generation 3.0;
wherein x is 1-500;
and wherein y is 1-32.

In embodiments, the compound may have the formula:

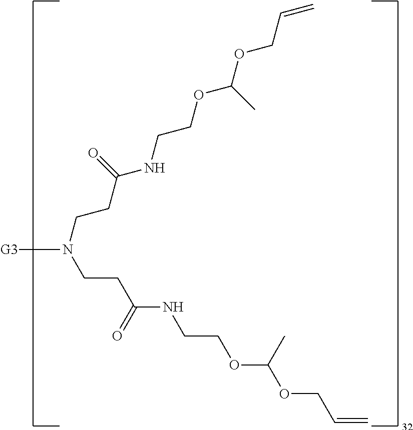

In embodiments, the compound may have the formula:

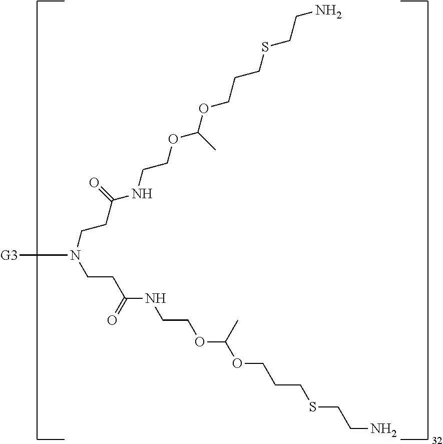

In embodiments, the compound may have the formula:

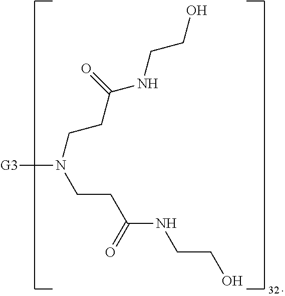

The ranges given for x independently include all values and sub-ranges therebetween. For example, x=1-500 includes 1, 10, 50, 80, 100, 114, 150, 170, 200, 227, 300, 400, 455, and 500. In one embodiment, x=1-50. In another embodiment, x=80-500. In another embodiment, x=1-100.

The ranges given for z independently include all values and sub-ranges therebetween. For example, z=1-500 includes 1, 10, 50, 80, 100, 114, 150, 170, 200, 227, 300, 400, 455, and 500. In one embodiment, z=1-150.

In embodiments, the y range depends on the generation of dendrimer. For example, y=1-128 includes 16, 32, 64, and 128 as appropriate, for G2, G3, G4, and G5 respectively.

Provided is a superior alternative to timolol eye drops or saline-based timolol eye drops, capable of maintaining decreased intraocular pressure with a less frequent dosing schedule. The compounds lower eye pressure longer than traditional timolol eye drops after a single dose.

In embodiments, provided are polyamidoamine (PAMAM) dendrimer-based compounds and compositions for the treatment of glaucoma and related diseases. Various embodiments provide compounds and compositions in which PAMAM dendrimer have beta-adrenergic antagonist moieties, commonly known as β-blockers, reduce IOP by slowing the production of aqueous humor.

In an embodiment, a dendrimer-based polymeric timolol analog is provided as glaucoma medication, which is highly water soluble and shows little or no signs of toxicity or ocular irritation in vitro or in vivo, and which has an IOP-lowering effect. The inventors have examined its PK/PD and studied how to use the multivalency of dendrimers to improve potency and safety. Optimization of drug loading and spacer length may if desired further improve its IOP-lowering effect.

In one embodiment, a dendrimer-based polymeric timolol analog as glaucoma medication, sometimes referred to herein as dendrimer-β-blocker conjugate DenTimol, may be prepared as follows; A timolol precursor (S)-4-[4-(oxiranylmethoxy)-1,2,5-thiadiazol-3-yl]morpholine (OTM) was reacted with the heterobifunctional amine polyethylene glycol acetic acid (Amine-PEG-Acetic Acid, Mₙ=2000 g/mol) via the ring opening reaction of epoxide by an amine to form OTM-PEG conjugate. OTM-PEG was then coupled to ethylenediamine (EDA) core polyamidoamine (PAMAM) dendrimer G3 to generate Dendrimer-β-blocker conjugate using the N-(3-dimethylaminopropyl)-N′-ethylcarbodiimide hydrochloride (EDC)/N-hydroxysuccinimide (NHS) coupling reaction. The reaction and intermediate and final products may be followed and characterized using MALDI mass spectroscopy, ¹H NMR spectroscopy, and HPLC. Ex vivo corneal permeation of dendrimer-β-blocker conjugate was assessed using the Franz diffusion cell system mounted with freshly extracted rabbit cornea. Cytotoxicity of dendrimer-β-blocker conjugate was assessed using WST-1 assay. Our results show that dendrimer-β-blocker conjugate is nontoxic up to an OTM equivalent concentration of 100 μM. Dendrimer-β-blocker conjugate is efficient crossing the cornea. About 8% of the dendrimeric drug permeated through the cornea in 4 h. Its IOP-lowering effect was observed in normotensive adult Brown Norway male rats. Compared to the undosed eye, an IOP reduction by an average of 7.3 mmHg (˜30% reduction from baseline) was observed in the eye topically treated with dendrimer-β-blocker conjugate in less than 30 min. Daily dosing of DenTimol for a week did not cause any irritation or toxicity as confirmed by the histological examination of ocular tissues including the cornea, ciliary body, and retina.

One embodiment provides a prodrug of timolol, a highly selective β blocker and anti-glaucoma drug, covalently coupled to a generation 3.0 PAMAM dendrimer through a permanent and flexible linker. Dendrimers have shown promise as drug delivery vehicles for other ocular drugs, but little work has been done with IOP lowering agents. OTM has similar bioactivity to timolol and is non-toxic, but it is highly hydrophobic. Coupling to a linear hydrophilic polymer such as PEG, can solubilize the compound, but as the present inventors have found, for example in the corneal permeability results, this structure does not readily penetrate the cornea. PAMAM dendrimers are also highly hydrophilic, but they are also compact and strongly cationic. It is believed that this unique architecture allows them to penetrate the cornea relatively easily even when covered with drug moieties.

The PEG spacer is believed to serve two purposes, it increases the bioactivity and reduces the toxicity of the drug-particle conjugate. PAMAM dendrimers are known to be cytotoxic at micromolar concentrations, but covering a portion of the particle's surface with PEG chains erases most of this effect.

The surface loading of the flexible spacer, or flexible spacer and β-blocker on the dendrimer is not particularly limiting, and 0.8-100% of the dendrimer surface is covered with the flexible spacer, or flexible spacer and β-blocker, or combination thereof. This range includes all values and subranges therebetween, including 0.8, 1, 10, 16, 25, 50, 60, 69, 80, 90 and 100% surface coverage. In one embodiment, about 60% of the dendrimer surface is covered with flexible spacer/β-blocker groups. In one embodiment, about 60% of the dendrimer surface is covered with PEG-OTM groups.

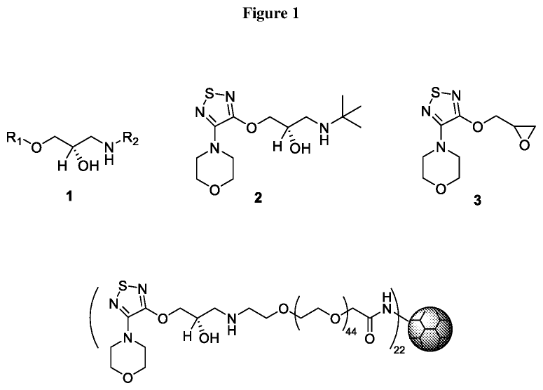

In structure 1 of common β-blockers (1), such as shown in FIG. 1, R₁ may be one of the following:

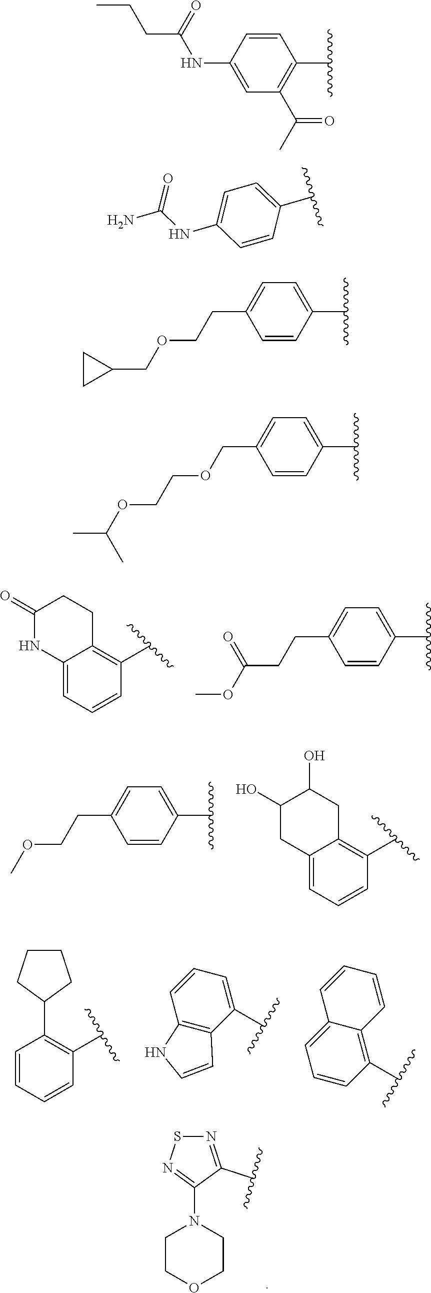

and R₂ in 1 may be —CH₃, —CH₂CH₃, —CH(CH₃)₂, or —C(CH₃)₃.

In one embodiment, R1 is

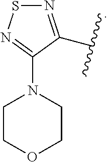 [SMILES: *c1nsnc1N1CCOCC1]

Animal tests showed that Dendrimer-β-blocker conjugate does lower IOP when administered in a topical drop. In one embodiment, the therapeutic window for timolol can be sufficiently extended to reduce dosing frequency to once a day or less.

One embodiment provides, for current antiglaucoma drugs or other small molecule drugs, or new drugs, an enabling delivery system to achieve sustained and effective delivery and improved bioavailability. In an embodiment, provided herein is a compelling proactive approach for antiglaucoma drug development and successfully build molecules with a known therapeutic effect or established therapeutic potential into a polymeric backbone to form pharmacologically active polymeric drugs. While many advances have been made in the development of polymeric drugs for some diseases, it is rarely used in the rational design of antiglaucoma drugs. The present approach can accelerate new antiglaucoma therapy development by capitalizing on benefits of polymeric drugs such as macromolecular multivalency and flexible structures allowing for accommodation of additional moieties to have desirable physicochemical properties for delivery. For the first time, a prodrug form of timolol is successfully directly coupled to a nanoparticle-based vehicle to increase its bioavailability and improve drug residence time. It is expected that other β-blockers such as those described herein could be similarly directly coupled to a nanoparticle-based vehicle to increase bioavailability and improve drug residence time.

Provided is a superior alternative to timolol eye drops or saline-based timolol eye drops, capable of maintaining decreased intraocular pressure with a less frequent dosing schedule. The compounds lower eye pressure longer than traditional timolol eye drops after a single dose.

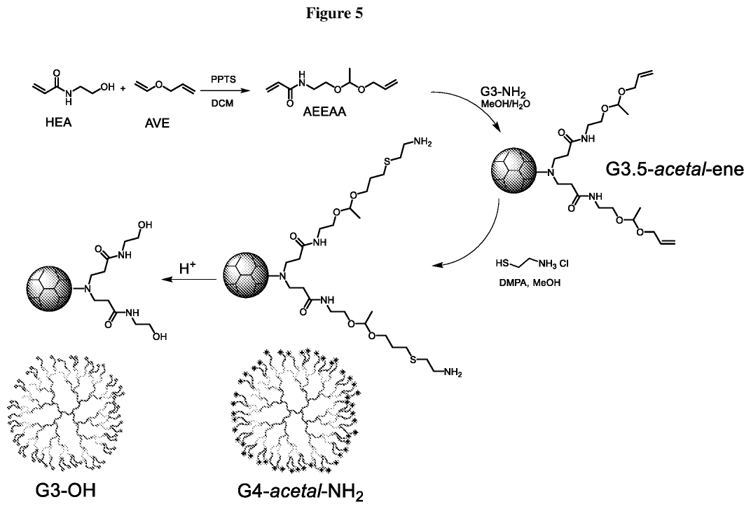

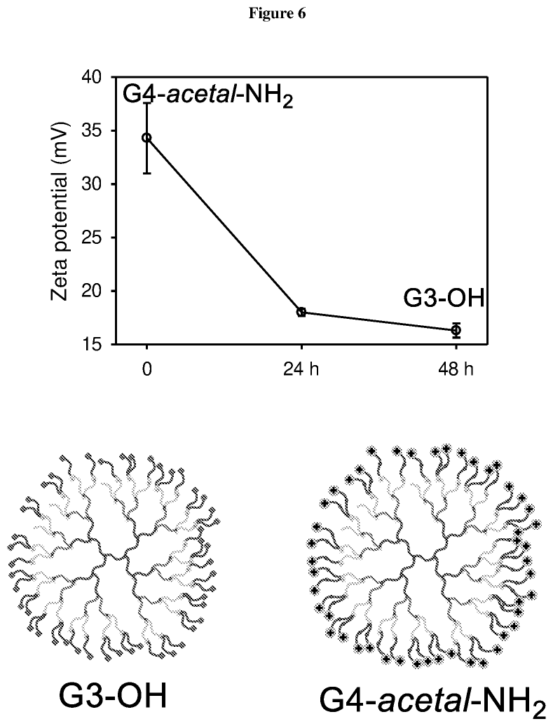

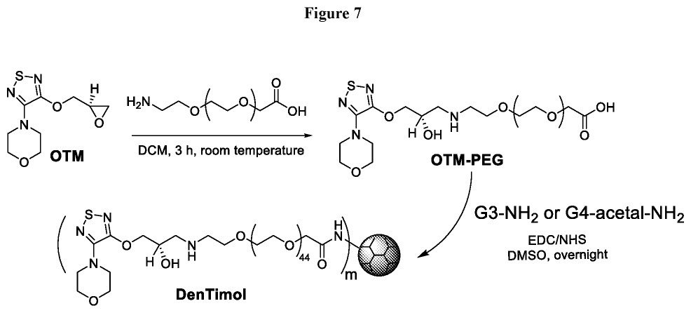

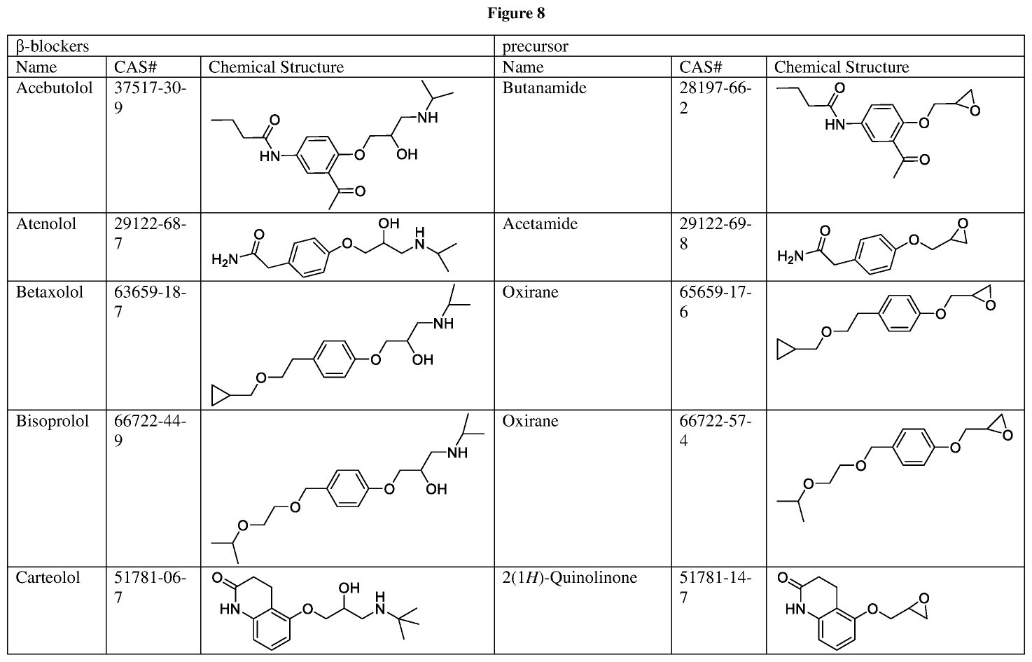

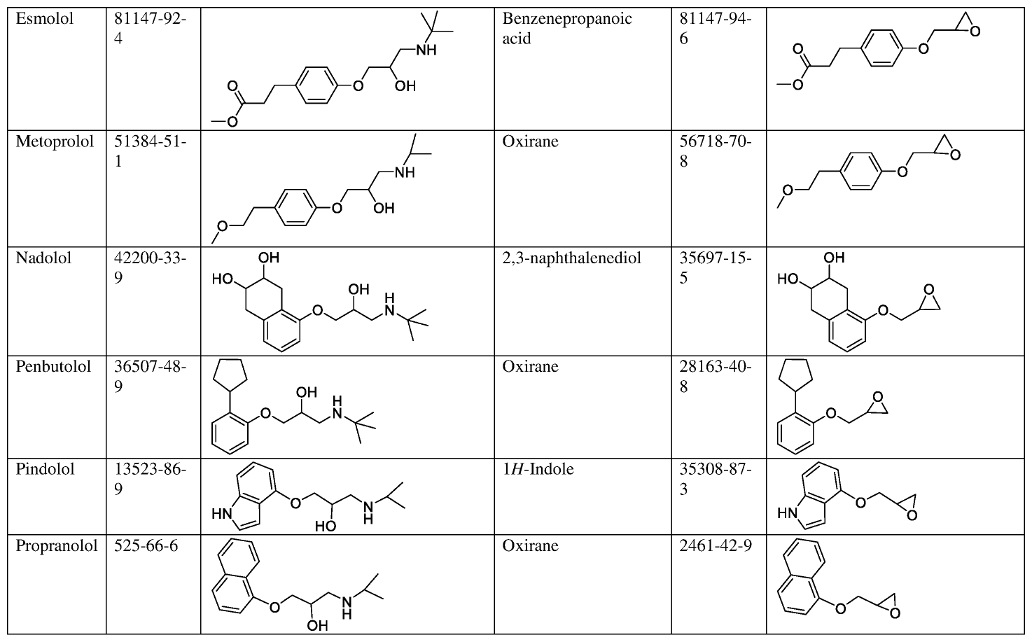

In one embodiment, a “smart” dendrimer carrier G4-acetal-NH₂ containing acid-labile acetal bonds is provided that can change the positive surface charge to neutral at various rates in a weakly acidic to a neutral environment (pH5-7.4) (FIG. 5 and FIG. 6). It is not uncommon that pH of aqueous humor changes from neutral to weakly acidic as a result of medication and pathogenesis of glaucoma. This new type still offers amine groups for dendrimer-β-blocker conjugate synthesis (FIG. 7).

The present inventors synthesized and characterized a dendrimer-based polymeric timolol analog (DenTimol) as a glaucoma medication. Efficacy and safety of this dendrimer-β-blocker conjugate were assessed in normotensive adult Brown Norway male rats. Developing IOP-lowering polymeric drugs is complementary to traditional small molecule antiglaucoma drug development and offers unique features by simultaneously tackling pharmacological potency and physiological barriers to delivery.

Some abbreviations, reagents, and background information-PAMAM (poly(amidoamine), a family of dendrimers); PEG (poly(ethylene glycol), a linear hydrophilic polymer); OTM (timolol prodrug); DCM (dichloromethane, an organic solvent); DMSO (dimethyl sulfoxide, a solvent miscible with water); EDC/NHS (N-(3-dimethylaminopropyl)-N′-ethylcarbodiimide hydrochloride and N-hydroxysuccinimide, these compounds facilitate the coupling of primary amines and carboxyl groups through a complex series of intermediates).

### EXAMPLES

The following examples are provided for better understanding and illustration, and are not intended to be limiting.

Synthesis. (S)-4-[4-(Oxiranylmethoxy)-1,2,5-thiadiazol-3-yl]morpholine (OTM) (Toronto Research Chemicals, Toronto, Canada, cat #0847080) was dissolved in dichloromethane (DCM) and reacted with an equimolar amount of amine polyethylene glycol acetic acid (Amine-PEG-Acetic Acid, Mₙ=2000 g/mol) (JenKem, Plano, Tex.) for 3 h at room temperature. The resulting OTM-PEG was recovered by rotary evaporation and purified by dialysis in water with a 3.5 kDa dialysis membrane and lyophilized. OTM-PEG (32 equiv.) was then reacted with EDA core polyamidoamine dendrimer generation 3.0 (G3.0) (1 equiv.) (Dendritech, Midland, Mich.) in DMSO overnight in the presence of a large molar excess of N-(3-dimethylaminopropyl)-N′-ethylcarbodiimide hydrochloride (EDC) and N-hydroxysuccinimide (NHS). The product DenTimol was purified by dialysis with a 7.5 kDa dialysis membrane for 48 h and lyophilized.

HPLC analysis. The reactants and products were analyzed with a Waters reverse phase HPLC system equipped with a Waters 2487 dual absorbance detector and an XTerra C18 column (4.6 mm×150 mm, particle size 5 μm). The mobile phase constituted water/acetonitrile (50:50, v/v). UV absorbance was monitored at 220 and 300 nm.

Matrix-assisted laser desorption/ionization mass spectrometry (MALDI MS). MALDI MS analysis was performed on an Applied Biosystems Voyager matrix-assisted laser desorption/ionization time-of-flight mass spectrometer. Spectra were calibrated internally for the AARS screening assay to 756.2352 (the mass of the 4-formylphenoxypropyl triphenylphosphonium AMP derivative). The reactant Amine-PEG-Acetic Acid and the intermediate OTM-PEG were dissolved in deionized water and spotted on a MALDI plate before analysis.

¹H NMR spectroscopy. ¹H NMR spectrum of DenTimol was collected on a Bruker 600 MHz NMR spectrometer. ¹H NMR (600 MHz, CD₃OD): δ (ppm) 3.89 (s, 2H), 3.80-3.73 (m, 4H), 3.73-3.59 (m, 223H), 3.52 (d, J=4.6 Hz, 2H), 3.47-3.35 (m, 5H), 3.27 (s, 3H), 3.18 (dd, J=10.0, 4.7 Hz, 2H), 2.85 (d, J=57.9 Hz, 9H), 2.60 (s, 3H), 2.38 (s, 7H).

Ex vivo corneal permeation study. Corneal permeability was determined using Franz diffusion cells (PermeGear, Hellertown, Pa.). Corneas were extracted from fresh rabbit eyes and placed immediately into diffusion cells with the endothelial surface facing the acceptor chamber and the epithelial surface facing the donor chamber. The acceptor chamber was filled with 5 mL of glutathione buffered Ringer's solution, and the donor chamber with 100 μL of a solution containing 1 mg of DenTimol (3.7 mM OTM equivalent). For comparison, permeation of OTM and OTM-PEG across the cornea were also tested at the same OTM equivalent concentration. The diffusion cells were placed in a water bath at 37° C. At various time points, 250 μL samples were withdrawn from the acceptor chamber, and drug concentrations were measured using the reverse phase HPLC system.

Cytotoxicity study. NIH 3T3 fibroblasts were seeded in a 96-well plate at 5000 cells per well and allowed 24 h to attach. The cells were maintained in DMEM medium supplemented with 10% serum, streptomycin (100 U/mL) and penicillin (100 U/mL) at 37° C. in 95% air/5% CO₂. Upon the removal of the medium, the cells were incubated with fresh DMEM medium (control) or fresh medium containing various amounts of OTM, OTM-PEG, or DenTimol (n=8) for 24 h. Cell viability relative to untreated cells was determined using the WST-1 cell proliferation assay.

In vivo efficacy assessment. Normotensive adult brown Norway rats (Charles River Labs, Wilmington Mass.) were used for all animal experiments in this study. They were housed under proper conditions at Virginia Commonwealth University (VCU). The rats were kept under a cycle of 12-h light and 12-h dark. The animal procedures were approved by the VCU IACUC. Drug efficacy was measured in vivo by dosing brown Norway rats (n=4) in the right eye with timolol solution (2×5 μL, 0.5% w/v) or an equivalent DenTimol solution. IOP was measured using a TonoLab rebound tonometer (Icare, Finland) at various time points in both eyes. Change in IOP was referenced to the contralateral (left) eye. No anesthetics or chemical restraints were employed during measurements. All measurements were taken by the same operator at the same location using the hand corresponding to that eye. Time 0 for all experiments (and baseline IOP readings) occurred at approximately 10 a.m. The reported IOP values are the average of 18-30 individual instrument readings of each eye.

In vivo safety assessment. To assess in vivo ocular tolerance, rats received OTM, timolol, or DenTimol (2×5 μL, 0.5% w/v OTM equivalent) in the right eye once daily for seven days. At the conclusion of the experiment, the rats were euthanized. The eyes were enucleated and immediately fixed in Davidson's solution, and 5 μm sections prepared for histological evaluation.

Statistical analysis. All the data are expressed as means±standard deviation. Student's t-test was performed for comparison. A value of p<0.05 was considered statistically significant.

### RESULTS AND DISCUSSION

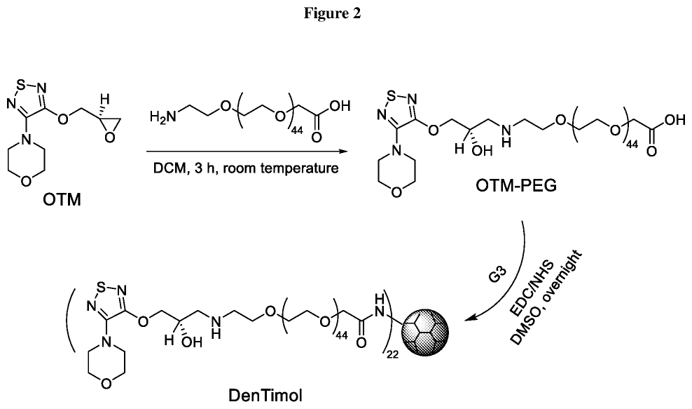

Synthesis and characterization. Most β-blockers contain 3-amino-1,2-propanediol (APD) (1 in FIG. 1), a key structural element responsible for antiglaucoma effects. Timolol (2) is a commonly prescribed β-blocker as a glaucoma medication. However, it lacks reactive groups that can directly conjugate with a polymer to form a polymeric antiglaucoma drug. To make a polymeric (dendrimer-based) timolol drug, we used a timolol precursor (5)-4-[4-(oxiranylmethoxy)-1,2,5-thiadiazol-3-yl]morpholine (OTM, 3), which has not only the same key chemical structure as timolol (highlighted in red) but also a reactive epoxy end-group (highlighted in blue) for functionalization. As shown in FIG. 2, the terminal amine group of the heterobifunctional PEG spacer Amine-PEG-Acetic Acid was reacted with the epoxy of OTM to generate OTM-PEG conjugate containing an APD moiety. MALDI mass spectrometry analysis shows that m/z value is 2057.7, suggesting a successful one-to-one coupling of OTM to PEG (PEG m/z=1880.0). OTM-PEG was then conjugated to PAMAM dendrimer G3 via the NHS/EDC coupling reaction. HPLC analysis confirmed that DenTimol was purified successfully and there was no unreacted OTM in the final conjugate. ¹H NMR spectrum shows that DenTimol has both PEG and OTM on the surface, and an average of 22 OTM-PEG were coupled to dendrimer according to integrals.

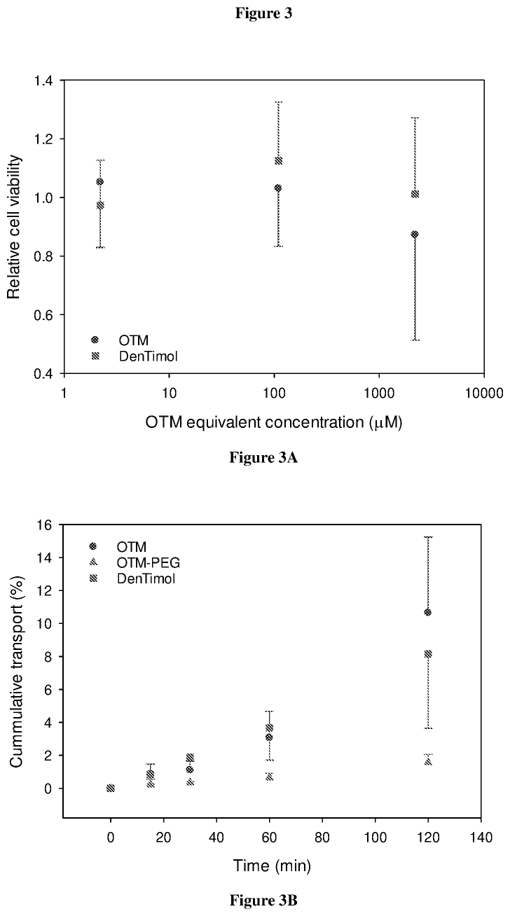

In vitro assessment. Covering a large portion of the dendrimer surface with PEG chains significantly improves dendrimer cytocompatibility. Indeed, our cytotoxicity results indicate that DenTimol shows no signs of cytotoxicity at the OTM equivalent concentration of 100 μM (FIG. 3A). Because of the high drug payload per dendrimer, this suggests DenTimol can be more effective in maintaining therapeutic concentrations for timolol.

OTM has similar bioactivity to timolol, but it is hydrophobic and has even lower ocular bioavailability than timolol maleate. Coupling to a linear hydrophilic polymer such as PEG can solubilize the compound, but as our corneal permeability results show this structure does not readily penetrate the cornea (<2% in 2 h) likely due to its high hydrophilicity (FIG. 3B). We found that DenTimol is efficient crossing the cornea: 8% of the drug permeated through the cornea in 4 h, which was 2.3-fold and 4-fold higher than timolol (3.5% in 4 h) and OTM-PEG (<2% in 2 h) (FIG. 3B), respectively. DenTimol's high corneal permeation is attributed to its hydrophilic-lipophilic balance with timolol being the outermost layer. PAMAM dendrimers are also highly hydrophilic, but they are also compact and strongly cationic. This unique architecture allows them to penetrate the cornea relatively easily even when covered with drug moieties. Direct coupling of OTM to dendrimer without a spacer is theoretically possible, and would likely result in an even higher level of bioavailability. However, without the flexibility provided by the polymeric linker, it is likely bioactivity of each prodrug moiety would be reduced, either due to steric hindrance from the dendrimer, or folding in of the dendrimer end groups towards the core of the molecule.

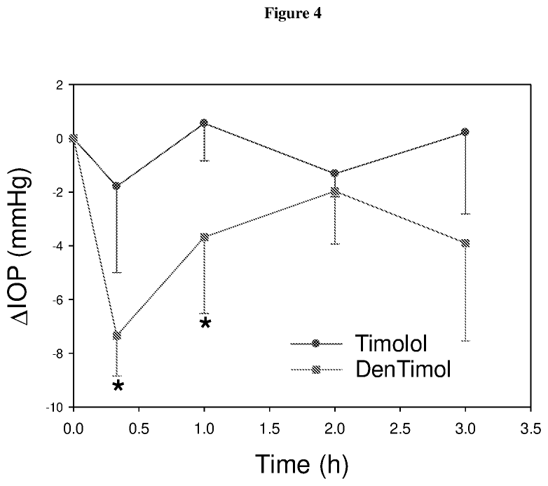

In vivo efficacy and safety assessment. One-time topical application of DenTimol (10 μl of 0.5% w/v timolol) in normotensive adult Brown Norway male rats resulted in an IOP reduction by an average of 7.3 mmHg (˜30%) in less than 30 min, which was significantly stronger than timolol PBS eye drops (FIG. 4) (both showed a peak effect in less than 30 min). The repeated application of DenTimol did not show any signs of toxicity or ocular irritation in vivo. H&E stained sections of ocular tissues confirmed that repeated dosing of DenTimol, OTM, or OTM-PEG did not alter the structures of the cornea, ciliary body, and retina (data not shown).

Shown in FIG. 5 is a smart surface charge-changing dendrimer under a weakly acidic environment. Step 1) AEEAA synthesis. HEA (12 equiv) reacts with AVE (10 equiv) in the PPTS (1 equiv)-containing DCM at ° C. for 0.5 h, followed by overnight reaction at room temperature overnight and then addition of excess K₂CO₃ to quench the reaction, and purification with flash chromatography in hexane/ethyl acetate (v/v=1/1). Step 2) G4-acetal-NH₂ synthesis. PAMAM dendrimer G3 (1 equiv) reacts with AEEAA (180 equiv) at 70° C. for 48 h, then reacts with cysteamine hydrochloride (140 equiv) in MeOH containing DMPA (1 equiv) under a 365 nm UV lamp for 4 h. Step 3) Acid-labile cleavage. Cleavage of the acetal groups changes highly positively charged G4-acetal-NH₂ to neutral G3-OH. Abbreviations: HEA, N-(2-hydroxyethyl) acrylamide; AVE, allyl vinyl ether; PPTS, pyridinium p-toluenesulfonate; DCM, dichloromethane; DMPA, 2-dimethoxy-2-phenylacetophenone; MeOH, methanol.

Shown in FIG. 7 is synthesis of dendrimer-β-blocker conjugate with modular functions. Either dendrimer G3 or G4-acetal-NH₂ can be used as underlying carrier. Abbreviations: DCM, dichloromethane; EDC, 1-Ethyl-3-[3-dimethylaminopropyl]carbodiimide hydrochloride; NHS, N-hydroxysuccinimide; DMSO, dimethyl sulfoxide.

Although the use of fully awake animals provides a more philologically relevant model, it produces more noise in the measurement of pressures by rebound tonometry. This noise makes it unclear at this point if potency is increased over timolol. The IOP response profile observed for dendrimer-β-blocker conjugate is roughly equivalent to literature reported values for timolol in rodents. The effect of chronic application was not studied. Clinical IOP lowering treatments rely on an additive effect of repeated drug dosing. We expect, at minimum, for this phenomenon to be present with dendrimer-β-blocker conjugate treatment if not greater due to the mucoadhesive quality of the dendrimeric drug. We believe dendrimer-β-blocker conjugate can extend the therapeutic window for timolol sufficiently to reduce dosing frequency to once a day or less. Dendrimer-β-blocker conjugate has three distinct structural features: 1) it retains the effective structure of timolol, thus gaining the potency as an antiglaucoma drug; 2) the incorporation of flexible polymeric spacers, for example PEG spacer can enhance the biocompatibility and water solubility of the new polymeric drug; 3) dendrimer-β-blocker conjugate is a novel self-transporting “smart” drug as PAMAM dendrimers can serve as a powerful ocular drug delivery vehicle with adaptable features and cornea transport into in the anterior chamber once applied to the eye.

A dendrimer-based polymeric timolol analog as glaucoma medication is provided. DenTimol is highly water soluble and shows no signs of toxicity or ocular irritation in vitro or in vivo. We observed its IOP-lowering effect, supporting us to fully examine its PK/PD and study how to use the multivalency of dendrimers to improve its potency and safety. Optimization of drug loading and spacer length may further improve its IOP-lowering effect.

1. A compound, comprising a selective β-blocker coupled to a PAMAM dendrimer via a flexible spacer.

2. The compound of claim 1, wherein the selective β-blocker is covalently bonded to the flexible spacer, and the spacer is covalently bonded to the PAMAM dendrimer.

3. The compound of claim 1, wherein the selective β-blocker is covalently coupled to the flexible spacer via a reaction of an oxirane group on the selective β-blocker.

4. The compound of claim 1, wherein the selective β-blocker is selected from the group consisting of Acebutolol, Atenolol, Betaxolol, Bisoprolol, Carteolol, Esmolol, Metoprolol, Nadolol, Penbutolol, Pindolol, Propranolol, Timolol, or combination thereof.

5. The compound of claim 1, wherein the selective β-blocker is timolol.

6. The compound of claim 1, wherein the PAMAM dendrimer is polyamidoamine dendrimer generation 2.0, 3.0, 4.0, or 5.0 (G2, G3, G4, or G5).

7. The compound of claim 1, wherein the flexible spacer is polyethylene glycol, polylactic acid, poly-L-lactic acid, poly(lactic-co-glycolic) acid, hyaluronic acid, or combination thereof.

8. The compound of claim 1, wherein the flexible spacer has one of the following formulas:
PLA (Polylactic acid), PLLA (Poly-L-lactic acid) 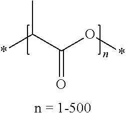
PLGA [Poly(lactic-co-glycolic acid)] 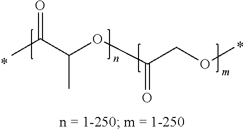
HA (Hyaluronic acid)

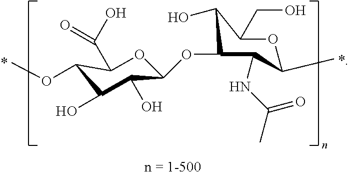

9. The compound of claim 1, wherein the flexible spacer is hydrolyzable.

10. The compound of claim 1, further comprising a plurality of the selective β-blockers coupled to the PAMAM dendrimer via a plurality of respective flexible spacers.

11. The compound of claim 1, having one of the following formulas: 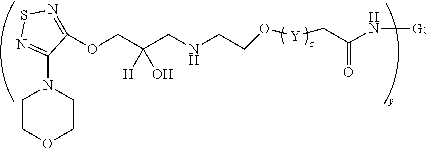
wherein Y is repeat unit of a flexible polymeric spacer;
wherein G is polyamidoamine dendrimer generation 2.0, 3.0, 4.0, or 5.0;
wherein y is 1-128; and
wherein z is 1-500.

12. The compound of claim 1, having one of the following formulas: 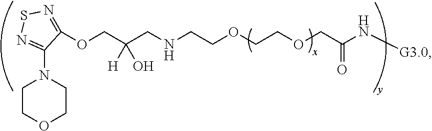
wherein G3.0 is polyamidoamine dendrimer generation 3.0;
wherein x is 1-500;
and wherein y is 1-32.

13. The compound of claim 1, having one of the following formulas: 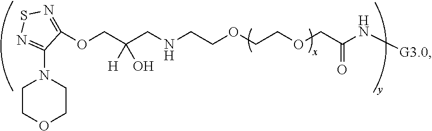
wherein G3.0 is polyamidoamine dendrimer generation 3.0;
wherein x is 44;
and wherein y is 22.

14. A pharmaceutical composition, comprising the compound of claim 1 and a physiologically acceptable carrier.

15. The composition of claim 14, wherein the physiologically acceptable carrier is an ophthalmic or ophthalmologically acceptable liquid carrier, solvent, diluent, emulsion, or dispersion.

16. A method of treating ocular hypertension or glaucoma, comprising administering the compound of claim 1 alone or in combination with a physiologically acceptable carrier to a subject.

17. A method, comprising carrying out the following reaction: 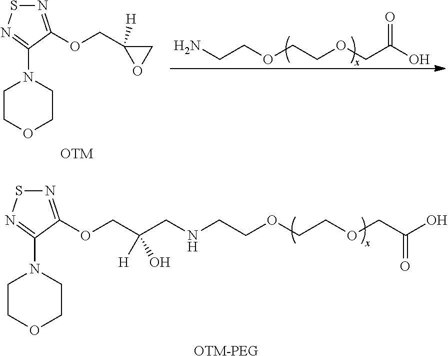
wherein x is 1-500.

18. A method, comprising carrying out the following reaction: 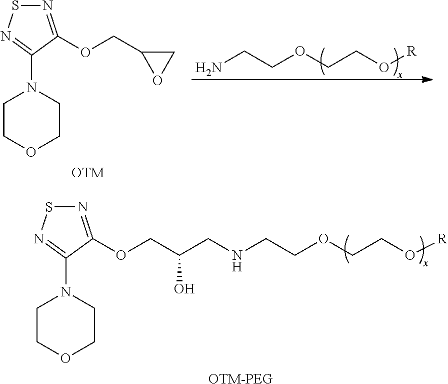
wherein x is 1-500; and
wherein R is one of the following:

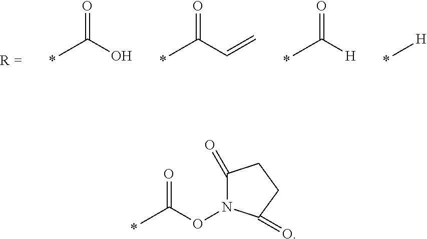

19. A method, comprising carrying out the following reaction: 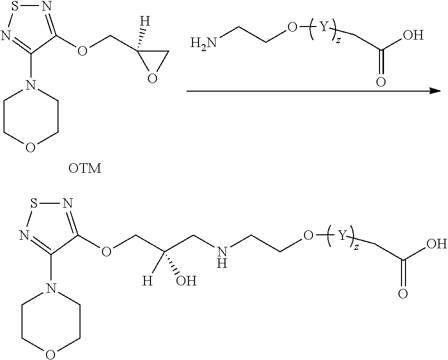
wherein Y is repeat unit of a flexible polymeric spacer; and
wherein z is 1-500.

20. A method, comprising carrying out the following reaction: 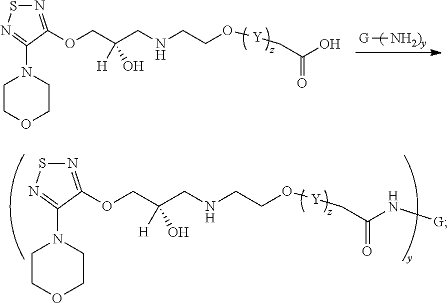
wherein Y is repeat unit of a flexible polymeric spacer;
wherein G is polyamidoamine dendrimer generation 2.0, 3.0, 4.0, or 5.0;
wherein y is 1-128; and
wherein z is 1-500.

21. The compound of claim 1, having one of the following formulas: 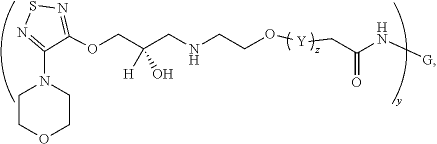
wherein Y is a flexible spacer;
wherein G is polyamidoamine dendrimer generation 2.0, 3.0, 4.0, or 5.0;
wherein y is 1-128; and
wherein z is 1-500.

22. A method, comprising carrying out the following reaction: 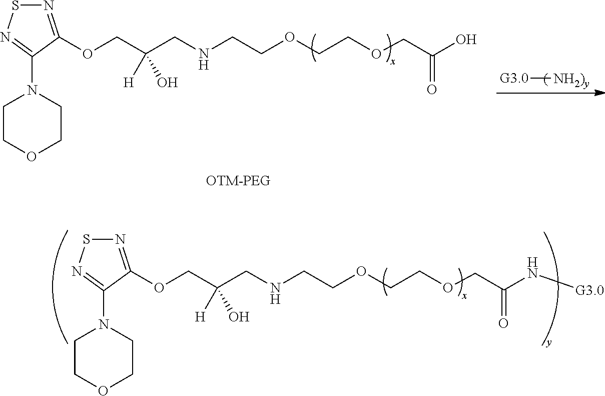
wherein G3.0 is polyamidoamine dendrimer generation 3.0;
wherein x is 1-500;
and wherein y is 1-32.

23. A method, comprising carrying out the following reaction: 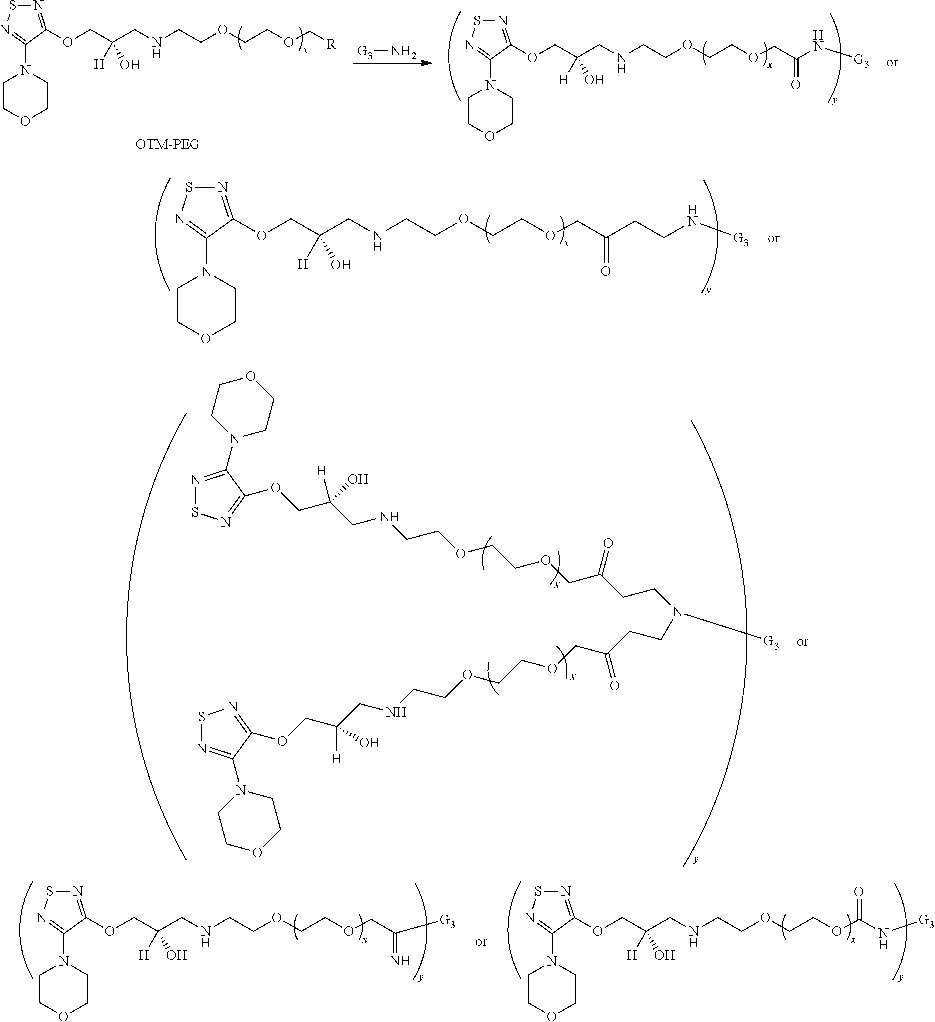
wherein G3.0 is polyamidoamine dendrimer generation 3.0;
wherein x is 1-500;
wherein y is 1-32; and
wherein R is one of the following:

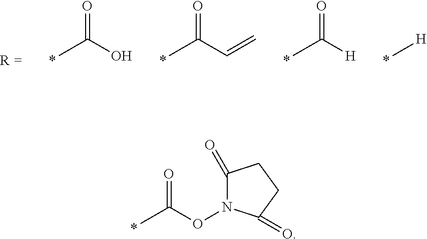

24. The method of claim 23, wherein x is 44 and y is 22.

25. A compound, having the following formula: 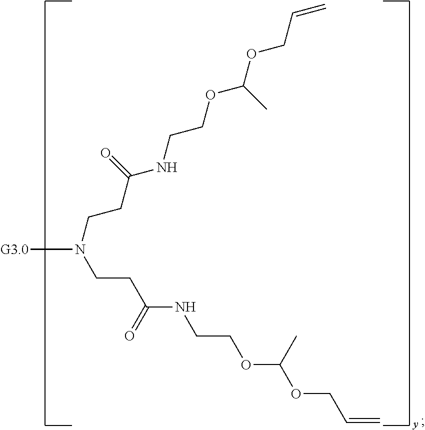
wherein G3.0 is polyamidoamine dendrimer generation 3.0;
and y is 1-32.

26. The compound of claim 25, having the formula:

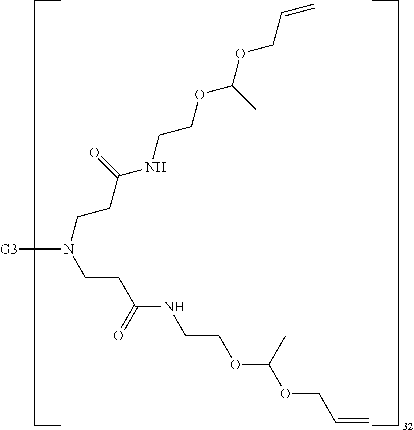

27. A compound, having the following formula: 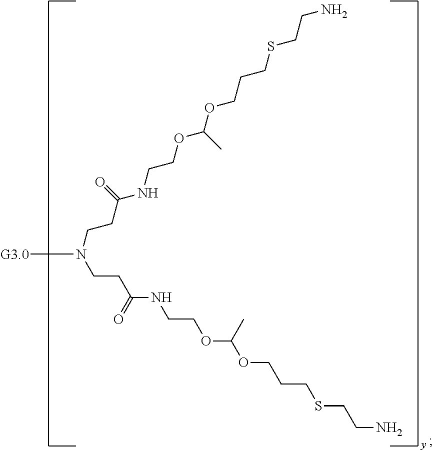
wherein G3.0 is polyamidoamine dendrimer generation 3.0;
and wherein y is 1-32.

28. The compound of claim 27, having the formula:

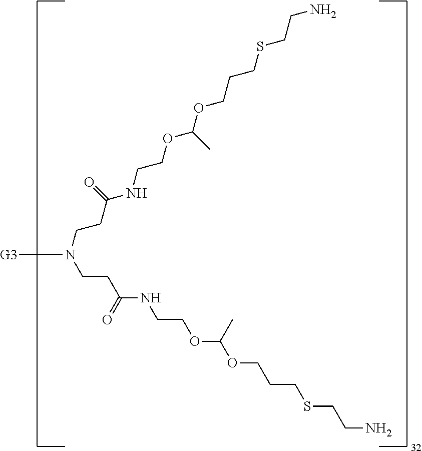

29. A compound, having the following formula: 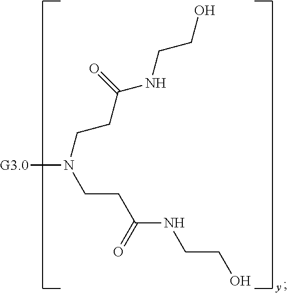
wherein G3.0 is polyamidoamine dendrimer generation 3.0;
and wherein y is 1-32.

30. The compound of claim 29, having the formula:

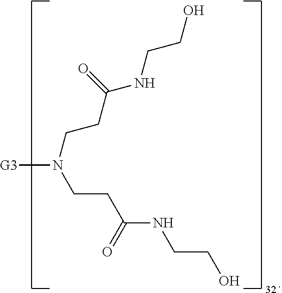

31. The compound of claim 1, having the following formula: 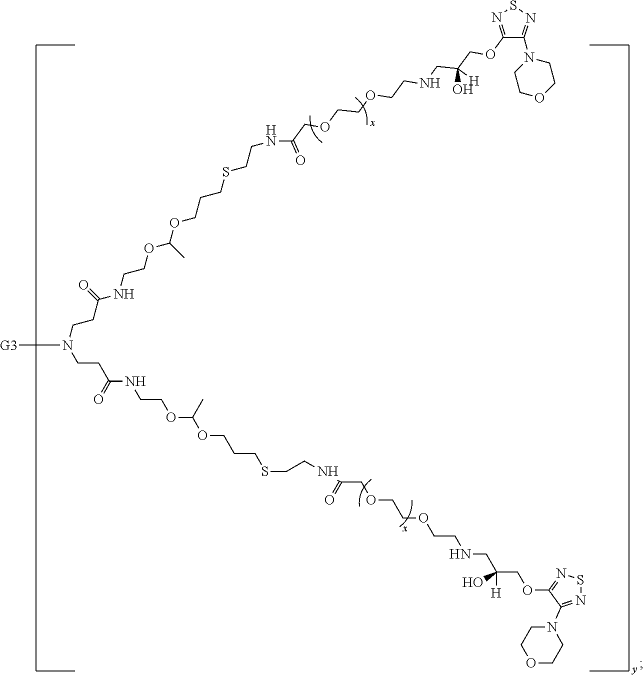
wherein G3.0 is polyamidoamine dendrimer generation 3.0;
wherein x is 1-500;
and wherein y is 1-32.

32. A method, comprising carrying out the following reaction: 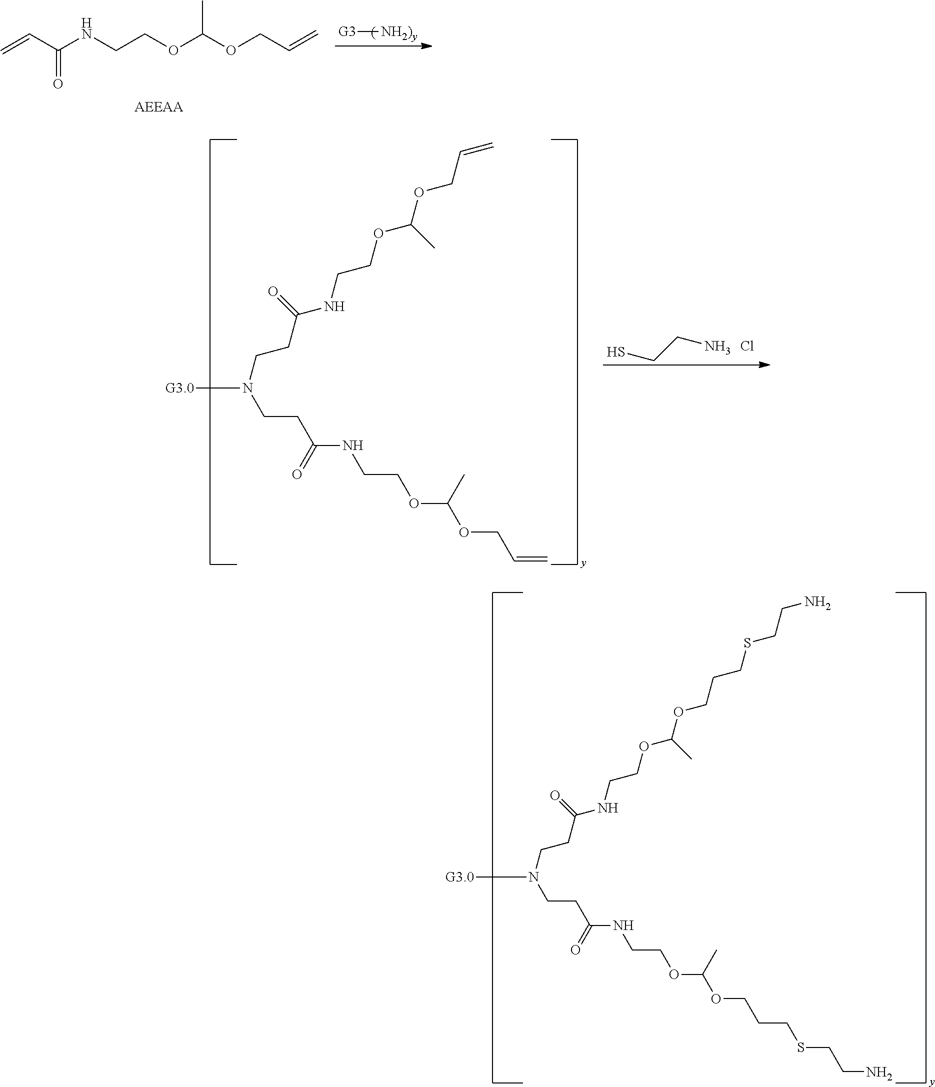
wherein G3.0 is polyamidoamine dendrimer generation 3.0;
and wherein y is 1-32.

33. A method, comprising reacting a pharmaceutically active compound, comprising an oxirane or epoxide group, with a flexible spacer and a PAMAM dendrimer, to form a pharmaceutically active dendrimer.
